(19) United States
(12) Patent Application Publication
(10) Pub. No.: US 2008/0213391 A1
(43) Pub. Date: Sep. 4, 2008

(54) Substance for the Treatment of Infections

(75) Inventors: Dirk Kaiser, Eppertshausen (DE); Mark-Andre Freyberg, Darmstadt (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Templa Tech GmbH, Darmstadt (DE)

(21) Appl. No.: 11/816,160

(22) PCT Filed: Feb. 10, 2006
(86) PCT No.: PCT/EP2006/001238

§ 371 Date: Aug. 13, 2007

(30) Foreign Application Priority Data
Feb. 12, 2005 (DE) 102005006462.0

(51) Int. Cl. A61K 33/00; A61P 31/00

(52) U.S. Cl. 424/600

(57) ABSTRACT

The invention relates to the use of dichloric acids and the derivatives thereof for producing medicaments used for disinfecting body cavities.

The mucous membranes of the external body orifices/cavities such as vagina, mouth, eyes, ears and nasal cavities are the natural habitat of many micro-organisms which are indeed indispensable for a healthy microbial microclimate. For example, lactic acid bacteria live on the mucosa of the vagina and their metabolite—lactic acid—acidifies the surrounding environment which helps to prevent infections of the mucosa by pathogens. Here however, as a result of many different causes, an invasion of pathogenic micro-organisms can still occur.

All micro-organisms which occur in internal body cavities must be considered to be intrinsically pathogenic because the inner cavities of the human body are normally germ-free.

Due to the increased resistance to antibiotics of the causative micro-organisms, the treatment of internal and external body cavity infections is often ineffective.

The cause of infections, for example, of the human bladder, is usually due to an invasion of bacteria via the urethra into the bladder space. The bladder itself is relatively insensitive to such pathogens but the situation can become highly problematical due to the risk of bladder infections ascending the ureter and reaching the kidneys.

This risk is particularly high in incontinent patients and elderly people as well as patients with traumatic alterations of the central nervous system who often also suffer from disturbed micturition. In many cases, these constellations lead to chronic urinary tract infections, which as a rule require permanent medication, which in its turn leads to further progressive resistance of the causative pathogens to antibiotics. The infection then ascends to affect the kidneys leading finally to renal failure.

Experience has shown that the treatment of these patents is very protracted and cost-intensive. For hospitalized patients, urinary tract infections considerably lengthen the stay in hospital which is both a psychological burden for the patients and a dramatic financial burden for the health care system.

The medium term risk of kidney damage and the long-term risk of high treatment expenditure can be reduced by eliminating the pathogens which have invaded the bladder via the urethra. This is especially important because renal insufficiency finally leads to dialysis treatment if not to organ transplantation.

After respiratory tract infections, urinary tract infections are the second most important infectious diseases. Within just one year, at least 20% of all women between the ages of 20 and 54 years suffer from a urinary tract infection (it is the most common reason of inability to work among women). Women are four times more often affected than men. Also, within the course of diabetes II, urinary tract infections are increasing considerably.

As a general rule, urinary tract infections in children always involve the risk of scar tissue formation in the renal parenchyma with possible late sequalae in adult life such as high blood pressure and renal failure. Therefore, all urinary tract infections in children should be treated urgently. The therapy period usually lasts between seven and fourteen days.

The so-called nosocomial infections have become a very large problem. (Infections are described as being nosocomial if they occur more than 48 hours after admittance to hospital and were not in the incubation phase at the time of admittance.) During a stay in hospital, 90% of all catheter wearers develop a urinary tract infection. Other groups of patients are also affected by nosocomial infections whereby urinary tract infections account for about 40%.

The development of resistance to pathogens is now a very important problem in the usual antibiotics treatment. Infections which were considered to have been overcome are again becoming a problem due to the occurrence of multi-resistant pathogens/strains. This multi-resistance is particularly due to the uncritical application of antibiotics.

The progress of modern medicine in recent years with larger numbers of older patients and more serious underlying conditions is accompanied by an increased danger of infections. In 1990, 30% of the patients in German hospitals were over 60 years of age (3.6 million).

Today, infections are among the most frequent complications in the treatment and care of older patients.

The risk of developing an infection in a nursing home is three times higher for older persons in comparison to younger people. Above the age of 65, the risk of developing a nosocomial infection is more than 20% higher and above the age of 75 it is more than 40% higher. These types of infections are an often underestimated problem in homes for geriatric patients. The incidence is reported as lying between 10% and 16%. The most common infections concerned the lower respiratory tract (bronchitis, pneumonia) with 49.7% and the urinary tract with 33.7%.

Problem bacteria, such as antibiotic resistant staphylococci (MRSA=methicillin-resistant Staphylococcus aureus), have become an important factor. In a study in 395 nursing homes for old people, MRSA strains were found in 12% of the patients admitted. Patients with MRSA were generally in a poorer condition and required more nursing care. In spite of the high rate of MRSA carriers however, the number of patients who actually contracted a disease was only between 3 and 4%. Infections which are acquired in nursing homes increase costs (in particular the costs for antibiotics and the costs of the sometimes long periods of intensive hospitalization which are necessary) and lead to increased morbidity and lethality rates.

The number of older patients who are incontinent and in need of care is steadily increasing. In view of the growing costs for looking after these patients, the necessity for an effective active substance—to which no resistance develops—to treat these patients is becoming increasingly important.

Another target group is also young children wearing diapers. Here, there is a considerable amount of urinary tract infections which can become a long-term hazard. Treatment without antibiotics is particularly desirable here.

Generally, women frequently suffer from urinary tract infections.

Further dangers are the nosocomial infections which in 40% of the cases lead to urinary tract disease. Particular catheters for discharging urine, for example, after operations, often lead to protracted urinary tract problems.

Chronically ill patients, in particular those with diabetes, often contract urinary tract infections. With increasing age, the number of male patients also increases sharply whereby women of all age groups are generally affected in much greater numbers. A further group of patients are pregnant women who suffer relatively frequently from urinary tract infections. Here, some of the traditional methods of treatment are considered problematical.

The dichloric acids and their manufacture were described in the not pre-published patent application number PCT/EP2004/013212. The use of the corresponding dichloric acids for promoting wound healing is also described there. The use as disinfectants was also generally described. The specific use as an agent for disinfecting body cavities, in particular those of the urogenital system, bladder, eyes, ears, nose, neck, throat and the open abdominal space during an operation was not revealed.

The object of the present invention therefore, was to develop a possibility of combating inflammation in/of body cavities. In particular, it should consist of a possibility with whose help antibiotic-resistant pathogens can also be combated. In this connection, the treatment should be simple and safe in its application.

Surprisingly, the above object can simply and reliably fulfilled by the use of an agent according to claim 1. Further embodiments of the present invention are shown in the independent and dependent claims 2 and 3.

The dichloric acids usable according to the present invention are shown in the following table 1. Among these dichloric acids, the dichloric acids No. 1 to No. 3 are especially preferred embodiments of the present invention.

TABLE 1

| | Formal oxidation | | |
|---|---|---|---|
| | numbers of | Structural formula of the | Structural formula of |
| No. | chlorine | acid | the dianion |
| 1 | +5, +5 | 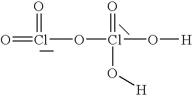 | 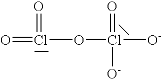 |
| 2 | +6, +4 | 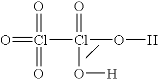 | 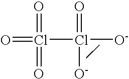 |
| 3 | +5, +5 | 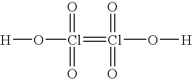 | 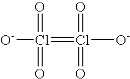 |
| 4 | +5, +3 | 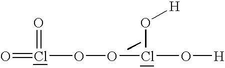 | 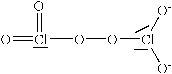 |

As well as the valence pairs +3/+5 (WO 00/48940) and +4/+4 (Bogdanchikov et al.) already described, the dichloric acids No. 1 to No. 3, usable according to the present invention, with the valences +6/+4 and +5/+5 for chlorine were manufactured for the first time with the method according to the present invention. The anion of the acid of No. 4 is described in WO 00/48940. The manufacturing method described there however, does not work.

WO 00/48940 postulated that the deoxo dimer is formed from 2 molecules of a reactive chlorine-oxygen species (peroxochlorate), via the reaction

2⁻OOCIO₂→CI₂O₆²⁻+O₂

whereby the chlorine atoms are present in the oxidation numbers +3 and +5. However, the manufacture of a stable compound according to embodiment 6 of WO 00/48940, which is desirable under pharmaceutical law aspects, is not successful.

The formation of the dimeric derivative from 2 molecules of peroxochlorate according to the formula

2⁻OOCIO₂→CI₂O₆²⁻+O₂

can namely only be expected at very high concentrations of peroxochlorate (from about 2 to 3 mol/l). Due to the high instability of the compound, such high concentrations are impossible to achieve in practice however.

Examinations show however, that the reaction of peroxochlorate ions O₂CIOO⁻ with chlorite ions (CIO₂⁻) leads surprisingly directly to the range of “dimeric” CI₂O₆²⁻ species:

O₂CIOO⁻+CIO₂⁻→CI₂O₆²⁻->->and isomers.

Insofar as ions are referred to in the present disclosure, the presence of the necessary counterions (particularly in solution) is also included. The designation “anions” is used in particular to underline the fact that, in solution, the dichlorate form is more stable compared with the protonated acid.

The invention also relates to the method for the manufacturing of preparations which contain the dichloric acids and their derivates, anions and/or salts.

In that:
(a) chlorine dioxide reacts with an aqueous or water-containing solution of hydrogen peroxide or another hydroperoxide or peroxide at a pH of ≧6.5,
(b) the pH is lowered by adding an acid
(c) the gaseous free protonated peroxochlorine compound is expelled with a cooled gas and collected in an alkaline solution,
(d) the collected peroxochlorine compound is incubated at a pH between 6 and 8, preferably at about 7 with up to 100-fold excess, preferably up to a 10-fold chlorite excess,
then, in this amazingly simple way, one can manufacture the dichloric acids usable according to the present invention.

The dichloric acids usable according to the present invention and also the anion which is present at physiological pH values can therefore, according to the present invention, also be present as a mixture with peroxochlorate and chlorite in solution. Such a solution, usable according to the present invention, containing dichloric acids, peroxochlorate and chlorite, therefore represents one of the particularly preferable embodiments of the present invention.

In contrast, in WO 00/48940, chlorite-free solutions were produced in which the dichloric acids of the present invention are not contained, or chlorite-containing preparations were produced which practically only contained chlorite which makes them unsuitable for use in pharmaceuticals.

The large amounts of chlorite however, are detrimental to the use of the dichloric acids in the pharmaceutical sector according to the present invention. Therefore, it is especially advantageous if the end-product of the solutions, according to the present invention, do not contain chlorite is more than 20-fold excess, preferably in not more than 5-fold excess and even more preferably in not more than a 3-fold excess in percentage by weight in relation to the total weight of the solution.

In particular, the dichloric acids, usable according to the present invention, are present in this solution in volumes of about 0.1-20 weight %, preferably 3-5 weight % in relation to the percentage by weight of the CIO₂ used. The qualitative identification is successful with Raman spectroscopy. Performing this type of spectroscopy is a matter of course for an expert in this field. The spectrograms which are obtained clearly differ from those which are obtained in the method described in WO 99/48940. The determination of the quantitative share of the dichloric acid can be carried out with acid-base titration.

A further qualitative detection is possible via the reaction with the heme iron. In the presence of the dichloric acids, usable according to the present invention, the temporal course of the change in intensity of the Soret bands is clearly different to the results with the solutions which were obtained with the method described in WO 00/48940.

The method according to the present invention consists of a reaction of chlorine dioxide with an aqueous or water-containing hydrogen peroxide (or another hydroperoxide or peroxide known to the expert, such as e.g. peroxocarbonate, or perborate or the urea adduct of the hydrogen peroxide) at a pH value of 6.5 or higher, preferably pH 10-12. It is preferred to maintain a constant pH level is desirable.

Moreover, surprisingly, it is evident that peroxochloric acid, which occurs as an intermediate product, as well as its anions and derivatives, can also be obtained by the reaction of chlorine dioxide with other oxidants which contain the peroxo group.

The reaction can be performed in an aqueous or water-containing environment. For example, as well as water, solvents can be present which are miscible with water—such as e.g. alcohols, such as e.g. alkanols such as methanol, ethanol or similar or mixtures of these.

As an alternative, other chlorine oxides can be used as the initial substance. For example, chlorine monoxide, preferably in its dimeric form (Cl₂O₂) can also be converted with a hydroperoxide (preferably hydrogen peroxide) to obtain the desired product. The reaction is successful at the same pH range as stated for chlorine dioxide.

The reaction temperature can be increased—for example up to about 50° C.; for purely aqueous systems, the lowest temperature should preferably be about 0° C. Furthermore, one should not utilize chlorine dioxide below +10° C. The chlorine dioxide gas liquefies below this temperature and deflagration can occur. If additional organic solvents and/or high concentrations of the active reagents are present, then one can also work with lower temperatures—i.e. below the freezing point of water. Room temperature is the preferred working temperature.

The chlorine dioxide needed for the reaction is available to experts and can be manufactured in the usual way. For example, it can be manufactured by the reaction of a chlorite with an acid (e.g. sodium chlorite with sulphuric acid) or by the reduction of chlorate, for example with sulphurous acid.

The chlorine dioxide thus obtained can be liberated in the usual way—if necessary after removal of traces of chlorine (Granstrom, Marvin L.; and Lee, G. Fred, J. Amer. Water Works Assoc. 50, 1453-1466 (1958).

If the chlorite used to make ClO₂ is contaminated with carbonate, the formation of ClO₂ occurs which is contaminated with CO₂ and/or the carbonic acid adducts described in WO 00/48940. In order to absorb the carbon dioxide, the gas stream containing chlorine dioxide and carbon dioxide should be directed through a washing bottle filled with lye. If there are only short contact times, the CO₂, but not the ClO₂, will be absorbed by the lye. It is better, however, to remove the carbonate contamination by fractioned crystallization of the sodium chlorite used. A contamination of the peroxochlorate with carbonate can be easily recognized from the Raman spectrum. Instead of the sharp band at 1051 cm⁻¹, a double band at 1069 cm⁻¹ (wide) as well as the important band, within the scope of the present invention, at 1051 cm⁻¹ (sharp) are obtained.

The chlorine dioxide can be transported with an inert gas such as nitrogen or with a rare gas such as argon, however, also with air or oxygen for the reaction with the peroxo compound or the hydroperoxide such as e.g. hydrogen peroxide, or percarbonate or perborate. For example, one can prepare the chlorine dioxide in a first reaction vessel and then to introduce it with the above mentioned gases or mixtures of them into a second reaction vessel which contains the peroxo compound (peroxide or hydroperoxide) in an aqueous or water-containing solution.

The pH value of the reaction mixture is kept equal to, or above, 6.5 by adding a base. It is preferable to keep the pH value constant. This can be carried out either manually or by using a “pH stat”.

The usual organic or inorganic bases can be employed, such as alkaline lyes, for example caustic soda solution or caustic potash solution or alkaline-earth hydroxides as well as ammonia, or organic bases such as nitrogenous bases. Also, the hydroxides from quaternary ammonium salts in particular alkyls, such as trialkyl- or tetraalkylammonium hydroxide, or zinc hydroxide can also be used.

The content of hydroperoxide in the reaction mixture can be determined, for example, using potentiometric titration with an acid such as e.g. hydrochloric acid.

The solutions obtained according to the methods described above can be employed either in the form in which they are made or in partial variations. For example, superfluous hydrogen peroxide can be removed in the usual way, e.g. with a heavy metal compound such as manganese dioxide. In a similar way, surpluses of the other oxidants can be also be removed.

Excess of chlorine dioxide (ClO₂) can be removed with H₂O₂. This should take place as quickly as possible, otherwise via

2ClO₂+2OH—->ClO₂—+ClO₃—+H₂O

disturbing ClO₃— will be formed. A product containing chlorate is however undesirable.

In order to improve the shelf life of the reaction product, storage at an increased pH value is recommendable, for example, pH 10 and above. The adjustment of this pH value can be carried out with a suitable base—as described previously above in the manufacturing method.

For the manufacture of solutions which contain the dichloric acids and/or the salts of these acids, surprisingly, it is possible to expel and trap the free acid HOOClO₂, the dichloric acids or the peroxochloric acid out of the mixture containing chlorite ions with an inert gas, e.g. a rare gas such as argon, or with nitrogen or with the gases oxygen or air, while lowering the pH to below 6, e.g. pH 5, or less. Surprisingly, it became evident that the yield can be increased enormously if the gas stream path is kept very short and the stream is cooled.

In the manufacturing procedure described above, the mixture which forms after the initiation of step (a) contains, at first, very high concentrations of chlorite ions (ClO₂⁻). The chlorite content, however, can be considerably reduced by “passing over” in a gas stream in a basic solution. In this process, all types of chloric acids are expelled as volatile compounds in protonated (neutral) form. These are very instable however. A base is contained in the receiving vessel, thus the chloric acids are deprotonated and the anions are formed. After the solution has been adjusted to pH 6-8, and after specific volumes of chlorite have been added—for example, as sodium chloride—the anions of the dichloric acids are formed.

Collection can be carried out, for example, in a base, such as an alkaline metal base, alkaline-earth metal base or a zinc base or nitrogenous base such as ammonia or an organic amine. It is also possible to freeze out the gaseous acids in a cold trap (e.g. at −100 to −190° C.).

The counterions which come into question are all metal cations and organic cations such as those from nitrogenous bases, in particular quaternary ammonium salts. The choice of the most suitable cations is determined by the individual purpose of use. Alkaline earth metals or alkaline metals, preferably Na⁺, K⁺, or Zn²⁺ are most suitable for pharmaceutical applications. In technical applications, organic cations, such as cations from nitrogenous bases, in particular alkylammonium cations such as trialkylammonium cations or especially quaternary ammonium cations can be used.

It is practical and preferable to store the acid and the salts in the dark and to make aqueous solutions with high pH values out of them, e.g. with pH values of 10, 11 or 12 and above, in particular the range of pH 10 to pH 13, in order to ensure a long storage life. Whenever required, the free acid can be regained from such solutions in the manner described previously and, if necessary, can be converted to solutions at the desired pH value or into salts.

The present invention also relates to pharmaceutical preparations and formulations which contain the dichloric acids or the anions, derivatives and salts thereof, according to the present invention, as the active substance, and which can be used particularly for the treatment of the inflammation/infections described in the introduction. The preparations contain the active substance alone or preferably with one or more pharmaceutically applicable carrier mediums or solvents (physiological saline, Ringer's solution etc.). The dosage of the active substance depends in particular on the location of the infection being treated (bladder, eye, abdominal space), as well as the species, age, weight and individual condition of the patient, individual pharmacokinetic factors as well as the method of application, the length of treatment and the treatment intervals.

In a preferred embodiment, 0.025 to 1 M solution of a dichloric acid salt and/or a salt of the derivatives thereof are dissolved in bidistilled water at a pH equal to or >10, preferably 10 to 13, in particular 12.5. Immediately before administration, this solution is diluted with common salt, sodium or potassium bicarbonate, phosphate buffer and bidistilled water to isotonie in concentrations of about 0.01 mM and adjusted to an almost physiological pH. Here, the final concentration for administration depends on the use.

The anions of the dichloric acids, usable according to the present invention, are stable but the acids themselves decompose relatively quickly so that the efficacy is reduced after about 14 days. Therefore, active substance stabilization can be carried out primarily by adjusting the pH. In order to improve tolerance, the active substance solution can be lowered to an almost physiological pH by buffer dilution immediately before administration. This is adequate for a deployment of the pharmacological action throughout the body, because this action does not rely on the receptor-ligand interaction of a conventional drug, but it is, as previously stated, related to a fast and irreversible oxidation reaction. Its pharmacological action remains in effect as long as the cell and/or its chemically changed structures are maintained, i.e. it is not terminated after diffusion of an active substance from a receptor.

In order to treat inflammation, the techniques of application of this agent are known to the expert. Here, depending on the location of the disease in the body, there are several possibilities. Because the agent according to the present invention is a solution, the easiest application technique is a rinse. For example, the expert will use a catheter for this purpose. In cases of an infection of the urogenital system, in particular the bladder, instillation is the preferable method.

For the prevention/prophylaxis of abdominal space infection, which frequently occurs, for example, as complications of operations on the gastrointestinal tract, the solutions is applied to the open wound before the closure of the abdominal wall. It remains there (disinfecting depot) and is simply “digested” so that a peritoneoclysis in the classical sense must not take place.

A particular advantage of the agent according to the present invention is that resistance development does not, indeed can not, take place.

Furthermore, most surprisingly, the agent according to the present invention, as well as its extremely good efficacy, showed no intolerance reactions and no allergic reactions whatsoever.

The instillation according to the present invention is the drop-by-drop application of liquid medications in the body (hollow organs, body cavities and orifices). Bladder instillation is a chemotherapeutical treatment of urinary tract infections. It can be performed with a syringe or applicator through an indwelling catheter (after previous catheterization).

Generally, the agent according to the present invention is excellent for disinfection purposes: for example, irrigating the nose and ears and rinsing any body cavity as well as application in the form of a “disinfecting depot” (e.g. abdominal space).

Furthermore, the agent according to the present invention is also excellent as a supporting measure in the standard therapy for infections of body cavities: for example bladder implants as revealed in the German patent application WO 2004/034930 A2.

The following embodiment examples provide more details about the invention, these should however by no means be understood in a limiting sense.

### EXAMPLE 1

### Preparation of the Dichloric Acids

Carefully, drop by drop, sulphuric acid (96%) is stirred into a solution of 100 g anhydrous sodium chlorite in 200 ml water. The chlorine dioxide which forms is expelled using a strong gas stream (Ar, N₂ or O₂ or CO₂-free air). The gas stream must be so strong that the content of ClO₂ does not exceed 5% (danger of explosion). In order to trap elemental chlorine, the gas stream containing ClO₂ is introduced into a consecutive series of three washing bottles which are each filled with 30 ml 2 M NaClO₂ solution at pH 11, in a solution of 15 ml of 30% hydrogen peroxide in 35 ml of water, which had previously been adjusted to pH 12 by adding 4M caustic soda solution. A solution of sodium perborate or sodium percarbonate or another peroxo compound, such as e.g. the H₂O₂ adduct of urea can be used instead of hydrogen peroxide. During the introduction of the gas, the pH value is controlled with a glass electrode. By adding 4M NaOH, the pH value during the reaction can be kept at 12. The hydroperoxide or the peroxo compound is exhausted when the inflow of gas leads to a permanent yellow coloring. A drop of the solution of the oxidant (e.g. H₂O₂) will subsequently decolorize the yellow solution again.

While stirring, the solution containing reactive chlorine is dripped into a solution of 500 g citric acid in 3 liters of water which has previously been adjusted to pH 4.5 with 2 M caustic soda solution. During this step, the reactive chlorine compound which forms is expelled with a strong gas stream (N₂ or O₂). Preferably, the gas stream should be cooled. The tube connections should be as short as possible. The gas is collected—for example in a consecutive series of three washing bottles which are each filled with 50 ml 0.1 M NaOH.

The contents of the three washing bottles are combined and kept at pH>10.

In order to form the dichloric acids usable according to the present invention, the pH is adjusted to 7—for example with hydrochloric acid—and a 10-fold molar excess of sodium chlorite is added.

The total content of reactive chlorine anions is determined by potentiometric titration with 0.1 M HCl with the usual method known to the expert.

The dichloric acids which are formed are present in solution in a mixture with a defined volume of chlorite as well as further reactive chlorine compounds.

The presence of the dichloric acids is detected with Raman spectroscopy.

### EXAMPLE 2

### Manufacture of the Pharmaceutical Preparation, According to the Present Invention, its Dosage and Application

The pharmaceutical preparation, according to the present invention, is manufactured by diluting the solution obtained from embodiment 1 in a pharmaceutical carrier medium (e.g. 0.9% NaCl solution, Ringer's solution, phosphate buffer etc) preferably in a ratio of 1:100 to 1:5,000.

Here, pharmaceutically suitable additives can also be included.

### EXAMPLE 3

### Treatment of Therapy-Resistant Bladder Infections

Treatment of patients with neurogenic micturition disturbances, e.g. due to SCI paralysis, with an exhaustively tested multiresistant bacterial spectrum.

The treatment of these patients is performed by instillation of the solution through a suprapubic indwelling catheter. Here, 50 ml of 1:2,000 diluted solution is instilled twice daily and remains in the bladder for about 30 minutes. After this, the irrigation solution is again discharged via the catheter. The length, concentration and volume of treatment can however vary and are determined by the physician with regard to the gravity of the disease and the general condition of the patient as well as other factors.

The daily irrigation cycles are carried out over a period of 5-7 days, at the discretion of the physician. The healing progress is controlled by testing and cystoscopy and the therapy is continued until no further evidence of infection can be detected.

1. (canceled)

2. The method of claim 10, wherein the treatment is in the form of an irrigation or a depot medication.

3. The method of claim 10, wherein the dichloric acid used as the active substance has the empirical formula H₂Cl₂O₆ and the following structural formulas of the anions.

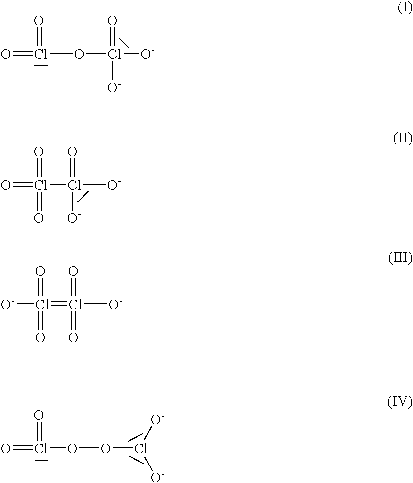

4. The method of claim 10, wherein the aqueous solution contains a concentration of a dichloric acid of at least 0.01 M.

5. The method of claim 10, wherein the dichloric acid is present in the form of at least one of the acid's alkaline metal, alkaline-earth metal, zinc, ammonia, and amine salts or derivatives thereof.

6. The method of claim 10, comprising collecting the free dichloric acid, or derivatives thereof in step (c), in a cold trap.

7. The method of claim 10, comprising introducing the free dichloric acid, or derivatives thereof from step (d) into an aqueous alkaline solution.

8. The method of claim 10, wherein an alkaline metal, alkaline-earth metal, zinc, or nitrogenous base or a hydroxide of a quaternary ammonium salt is used as the base in the basic solution in step (c).

9. The method of claim 10, comprising stabilizing the solutions obtained from step (d) by increasing the pH value.

10. Method of anti-infectious treatment of a body cavity, said method comprising the step of treating the body cavity with at least one of a medication or pharmaceutical preparation comprising as an active substance at least one dichloric acid or physiologically tolerated derivative or salt thereof, said dichloric acid being manufactured by a process comprising the steps of:
(a) reacting chlorine dioxide with an aqueous or water-containing solution of hydrogen peroxide or another hydroperoxide or peroxide at a pH value of at least 6.5;
(b) lowering the pH value to 3 to 6 by adding an acid;
(c) expelling a gaseous free reactive peroxo chlorine compound produced thereby with a cooled gas and trapping the expelled peroxo chlorine compound in a basic solution with a pH value greater than 10; and,
(d) incubating the trapped peroxo chlorine compound with up to 100-fold excess of chlorite at a pH of 6 to 8 to obtain said dichloric acid.

11. The method of claim 10 wherein the body cavity is selected from the group consisting of abdominal cavities, nasal cavities, paranasal cavities, the ear, and the mouth.

12. The method of claim 10 wherein the body cavity is the urinary bladder.

13. The method of claim 10 wherein the peroxo chlorine compound is incubated with an up to 10-fold excess of chlorite.

14. The method of claim 10 wherein the peroxo chlorine compound is incubated with chlorite at a pH of about 7.

15. The method of claim 10 wherein the peroxo chlorine compound is incubated with an up to 10-fold excess of chlorite at a pH of about 7.

16. The method of claim 10, wherein the aqueous solution contains a concentration of a dichloric acid of at least 0.025 M.

17. The method of claim 10, wherein the aqueous solution contains a concentration of a dichloric acid of at least 0.05 M.

18. The method of claim 10, wherein the aqueous solution contains a concentration of a dichloric acid of at least 0.075 M.

19. The method of claim 10, wherein the aqueous solution contains a concentration of a dichloric acid of at least 0.1 M.

20. The method of claim 10, wherein the aqueous solution contains a concentration of a dichloric acid of at least 0.5 M.

21. The method of claim 10, wherein the dichloric acid has structural formula I.

22. The method of claim 21, wherein the dichloric acid has structural formula II.

23. The method of claim 21, wherein the dichloric acid has structural formula III.
